(19) United States
(12) Patent Application Publication
(10) Pub. No.: US 2026/0240795 A1
(43) Pub. Date: Aug. 20, 2026

(54) SEPHIN1 AND RELATED COMPOUNDS FOR THE TREATMENT OF DISEASES AND DISORDERS AMENABLE TO TREATMENT BY AMPK ACTIVATION AND/OR CHOP INHIBITION

(71) Applicant: KYOTO PREFECTURAL PUBLIC UNIVERSITY CORPORATION, Kyoto (JP)

(72) Inventor: Satoshi GOJO, Kyoto (JP)

(73) Assignee: KYOTO PREFECTURAL PUBLIC UNIVERSITY CORPORATION, Kyoto (JP)

(21) Appl. No.: 19/164,898

(22) PCT Filed: Mar. 15, 2024
(86) PCT No.: PCT/IB2024/052531

§ 371 Date: Sep. 12, 2025

Related U.S. Application Data
(60) Provisional application No. 63/490,538, filed on Mar. 16, 2023.

(51) Int. Cl. A61K 31/155

(52) U.S. Cl. CPC A61K 31/155

(57) ABSTRACT

Methods of treating diseases and disorders treatable by AMPK activation with sephin1 and related compounds, for example metabolic syndrome, non-alcoholic fatty liver disease, nonalcoholic steatohepatitis (NASH), obesity, type 2 diabetes, insulin resistance, glucose intolerance, chronic pain, sarcopenia, a neuromuscular disorder, heart failure, cardiac hypertrophy, diabetic cardiomyopathy, cardiac reperfusion injury, chronic kidney disease, polycystic kidney disease, diabetic nephropathy, cardiovascular diseases, inflammatory bowel disease, arthritis, hypertension, peripheral vascular disease, nephrogenic diabetes insipidus, glaucoma, an eye disease or disorder, ocular neovascularization, β-hemoglobinopathy, cancer, fibrosis, cancer cachexia, and mitochondrial disease.

### 1. CROSS-REFERENCE TO RELATED APPLICATIONS

This application claims the priority benefit of U.S. provisional application No. 63/490,538, filed Mar. 16, 2023, the contents of which are incorporated herein in their entireties by reference thereto.

### 2. BACKGROUND

Sephin1 (also known as IFB-088 and icerguastat) is a benzylideneguanidine derivative under development for treatment of amyotrophic lateral sclerosis (ALS) and Charcot-Marie-Tooth disorder (CMT).

### 3. SUMMARY

This disclosure is based, in part, on the discovery that the compound sephin1 activates AMP-activated protein kinase (AMPK). AMPK is a regulator of energy homeostasis and increased AMPK activity is associated with a variety of health benefits (Steinberg & Carling, 2019, Nat. Rev. Drug Discov. 18(7):527-551). Activated AMPK phosphorylates C/EBP homologous protein (CHOP), an endoplasmic reticulum (ER) stress-induced transcription factor. Phosphorylation of CHOP by AMPK triggers the proteosomal degradation of CHOP (Dai et al., 2016, Circ Res. 119(10):1089-1100). AMPK is a known target for treating various diseases and disorders such as metabolic syndrome, type-2 diabetes, and cancer (Li et al., 2015, Oncotarget 6(10):7365-7378), and CHOP is also implicated in various diseases and disorders including cancer, diabetes, and fibrosis (Yang et al., 2017, Frontiers in Immunology 8:1612). CHOP also regulates GDF-15, which plays a role in lipid hypermetabolism (Li et al., 2019, Biochem Biophys Res Commun 498(3):388-394, Townsend et al., 2022, J Appl Physiol 132: 413-422). Lipid hypermetabolism is associated with diseases and disorders including cancer cachexia, advanced cancer, and mitochondrial disease (Sturm et al., 2023, Communications Biology 6:22).

Thus, the disclosure provides new uses for sephin1 and related compounds, for example in the treatment of diseases and disorders treatable by AMPK activation and/or treatable by CHOP inhibition.

In one aspect, the disclosure provides a method of activating AMPK and/or increasing phosphorylated AMPK (pAMPK)/AMPK ratio in a cell by contacting a cell of a subject (e.g., a cell in vivo) with an agent which is a compound of Formula (I)

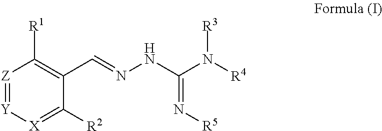

or a tautomer thereof, or a pharmaceutically acceptable salt of the foregoing, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms (such as N) in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy;
X and Z are each independently CR¹¹
Y is CR¹¹ or N; and
R¹¹ is H, alkyl or F.

Sephin1 is an exemplary compound of Formula (I).

Compounds of Formula (I), tautomers thereof, and pharmaceutically acceptable salts of the foregoing (for avoidance of doubt, “pharmaceutically acceptable salts of the foregoing” refers to pharmaceutically acceptable salts of compounds of Formula (I) and pharmaceutically acceptable salts of tautomers of compounds of Formula (I)) are sometimes collectively referred to herein as “agents of the disclosure” for convenience.

In another aspect, the disclosure provides a method of inhibiting CHOP increasing the phosphorylated CHOP (pCHOP)/CHOP ratio in a cell by contacting a cell of a subject (e.g., a cell in vivo) with an agent of the disclosure.

In another aspect, the disclosure provides a method of treating a subject having or at risk of disease or disorder treatable by AMPK activation and/or treatable by CHOP inhibition, the method comprising administering an agent of the disclosure to the subject. Exemplary diseases and disorders treatable by AMPK activation and/or inhibiting CHOP include metabolic syndrome, non-alcoholic fatty liver disease, nonalcoholic steatohepatitis (NASH), obesity, type 2 diabetes, insulin resistance, glucose intolerance, chronic pain, sarcopenia, a neuromuscular disorder, heart failure, cardiac hypertrophy, diabetic cardiomyopathy, cardiac reperfusion injury, chronic kidney disease, polycystic kidney disease, acute kidney injury, diabetic nephropathy, cardiovascular diseases, inflammatory bowel disease, arthritis, hypertension, peripheral vascular disease, nephrogenic diabetes insipidus, glaucoma, an eye disease or disorder, ocular neovascularization, β-hemoglobinopathy, cancer, and fibrosis. Further exemplary diseases and disorders treatable by AMPK activation and/or inhibiting CHOP include cancer cachexia, advanced cancer, mitochondrial diseases, and amyotrophic lateral sclerosis (ALS).

In some embodiments of the methods of the disclosure, the agent is sephin1 or a tautomer thereof, a salt of sephin1, or a salt of a tautomer of sephin1.

Methods of the disclosure are further described in Section 5.2 and numbered embodiments 1 to 105, infra.

Agents of the disclosure and compositions comprising the agents are further described in Section 5.3 and numbered embodiments 1 to 108, infra.

### 4. BRIEF DESCRIPTION OF THE FIGURES

FIG. 1 shows the study timeline of Example 1.

FIG. 2 shows Western blots for phosphorylated AMPK (pAMPK), AMPK, and GAPDH (control) from HK-2 cells treated with Tunicamycin (Tu), Tunicamycin+Sephin1 (Tu+Se) or untreated cells (CNT) (Example 1).

FIGS. 3A-3C show pAMPH/GAPDH (FIG. 3A), AMPK/GAPDH (FIG. 3B) and pAMPK/AMPK (FIG. 3C) ratios from HK-2 cells treated with Tunicamycin (Tu), Tunicamycin+Sephin1 (Tu+Se) or untreated cells (Sham) (Example 1).

FIG. 4 shows the study timeline of Example 2.

FIG. 5 shows protein expression levels for CHOP, p-CHOP, and α-Tubu treated with Tunicamycin (Tu), Tunicamycin+Sephin1 (Tu+Se) or untreated cells (Sham) (Example 2).

FIG. 6 shows relative CHOP, p-CHOP and p-CHOP/CHOP levels from the study of Example 2.

FIG. 7 shows the study timeline of Example 3.

FIG. 8 shows protein expression levels and relative protein expression levels for CHOP, p-CHOP, and α-Tubulin from cells treated with Tunicamycin (Tu), Tunicamycin+Sephin1 (Tu+Se) or untreated cells (Sham) (Example 3).

FIG. 8 shows protein expression levels and relative protein expression levels in cytoplasm and nucleus for CHOP, α-Tubulin, and histone from cells treated with Tunicamycin (Tu), Tunicamycin+Sephin1 (Tu+Se) or untreated cells (CNT) (Example 3).

FIG. 9 shows cell number and cell viability for the treatment groups of Example 3.

### 5. DETAILED DESCRIPTION

### 5.1. Definitions

Unless defined otherwise, all technical and scientific terms used herein have the same meaning as commonly understood to one of ordinary skill in the art to which this invention belongs. The following definitions are provided for the full understanding of terms used in this specification.

As used herein, the following terms are intended to have the following meanings:
A, An, The: As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein.
Alkenyl: As used herein, the term “alkenyl” refers to a group containing one or more carbon-carbon double bonds, and which can be branched or unbranched. An alkenyl group can be, for example, a C₂₋₂₀ alkenyl group, a C₂₋₁₅ alkenyl group, a C₂₋₁₂ alkenyl group, a C₂₋₆ alkenyl group, or a C₂₋₃ alkenyl group. The term “cyclic alkenyl” is to be construed accordingly.
Alkyl: As used herein, the term “alkyl” includes both saturated straight chain and branched alkyl groups. An alkyl group can be, for example, a C₁₋₂₀ alkyl group, a C₁₋₁₅ alkyl group, a C₁₋₁₂ alkyl group, a C₁₋₆ alkyl group, or a C₁₋₃ alkyl group. Examples of include methyl, ethyl, propyl, isopropyl, butyl, isobutyl, tert-butyl, pentyl and hexyl.
AMPK: The term “AMPK” refers to AMP-activated protein kinase. AMPK is a heterotrimeric protein complex that is formed by α, β, and γ subunits. The α, β, and γ subunits can be found in different isoforms: the γ subunit can exist as the γ1, γ2 or γ3 isoform; the β subunit can exist as the β1 or β2 isoform; and the α subunit can exist as the α1 or α2 isoform. The a subunits are encoded by the PRKAA1 (α1 isoform) and PRKAA2 (α2 isoform) genes; the β subunits are encoded by the PRKAB1 (β1 isoform) and PRKAB2 (β2 isoform) genes, and the γ subunits are encoded by the PRKAG1 (γ1 isoform), PRKAG2 (γ2 isoform), and PRKAG3 (γ3 isoform) genes. AMPK becomes activated when phosphorylation takes place at Thr-172 of the a subunit (Sanders et al., 2007, Biochem J. 403(Pt.1):139-148; Steinberg & Carling, 2019, Nat. Rev. Drug Discov. 18(7):527-551). Phosphorylated AMPK is referred to herein as “pAMPK.”
And/or: The term “and/or” means that each one or both or all the components or features of a list are possible variants, especially two or more thereof in an alternative or cumulative way.
Aralkyl: As used herein, the term “aralkyl” refers to a group having both aryl and alkyl functionalities. By way of example, the term includes groups in which one of the hydrogen atoms of the alkyl group is replaced by an aryl group, e.g. a phenyl group. Exemplary aralkyl groups include benzyl, phenethyl and the like.
Aryl: As used herein, the term “aryl” refers to a C₆-1₂ aromatic group. Examples include phenyl and naphthyl, etc.
CHOP: The term “CHOP” refers to C/EBP homologous protein. CHOP is a proapoptotic transcription factor that is encoded by the DDIT3 gene and induced in response to certain stressors (Yang et al., 2017, Frontiers in Immunology 8:1612). Phosphorylation of Ser-30 of CHOP by AMPK triggers the proteosomal degradation of CHOP (Dai et al., 2016, Circ Res. 119(10):1089-1100). CHOP phosphorylated at Ser-30 is referred to herein as “phosphorylated CHOP,” “pCHOP,” or “p-CHOP.” The expressions “inhibiting CHOP,” “CHOP inhibition” and the like do not require total inhibition of CHOP. Inhibition can be achieved, for example, by increasing the amount of pCHOP and/or increasing a pCHOP/CHOP ratio and/or reducing the amount of CHOP, for example due to proteosomal degradation of pCHOP.
Cycloalkyl: As used herein, the term “cycloalkyl” refers to a cyclic alkyl group.
Effective amount: The term “effective amount” or “therapeutically effect means the amount or quantity of an agent or composition that is sufficient to elicit the required or desired response, or in other words, the amount that is sufficient to elicit an appreciable biological response when administered, e.g., to a subject. Said amount preferably relates to an amount that is therapeutically or in a broader sense also prophylactically effective against the progression of a disease or disorder as disclosed herein. It is understood that an “effective amount” or a “therapeutically effective amount” can vary from subject to subject, due to variation in metabolism of an agent, age, weight, general condition of the subject, the condition being treated, the severity of the condition being treated, and the judgment of the prescribing physician.
Heteroaryl: As used herein, the term “heteroaryl” refers to a 4 to 12 membered aromatic group, which comprises one or more heteroatoms. An exemplary heteroaryl group is a 4 to 12 membered aromatic group comprising one or more heteroatoms selected from N, O and S. Exemplary heteroaryl groups include pyrrolyl, pyrazolyl, pyrimidinyl, pyrazinyl, pyridinyl, quinolinyl, thiophenyl, 1,2,3-triazolyl, 1,2,4-triazolyl, thiazolyl, oxazolyl, isothiazolyl, iso-oxazolyl, imidazolyl, furanyl and the like.
Heterocycle: As used herein, the term “heterocycle” (also referred to herein as “heterocyclyl” and “heterocyclic”) refers to 4 to 12 membered, for example 4 to 12 membered saturated, unsaturated or partially unsaturated, cyclic group containing one or more heteroatoms selected from N, O and S, and which optionally further contains one or more CO groups. The term “heterocycle” encompasses both heteroaryl groups and heterocycloalkyl groups as defined herein.
Heterocycloalkyl: As used herein, the term “heterocycloalkyl” refers to a 4 to 12 membered cyclic aliphatic group which contains one or more heteroatoms. Exemplary heterocycloalkyl groups include piperidinyl, pyrrolidinyl, piperazinyl, thiomorpholinyl and morpholinyl. Further examples include N-piperidinyl, N-pyrrolidinyl, N-piperazinyl, N-thiomorpholinyl and N-morpholinyl.
Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.
Subject: As used herein, the term “subject” means a human.
Treat, treating, treatment: The terms “treat,” “treating,” “treatment,” anc variations thereof as used herein, include reducing or ameliorating a disease or disorder, and/or signs or symptoms associated therewith, or slowing or halting the progression thereof. It will be appreciated that, although not precluded, treating a disease or disorder does not require that the disease, disorder or symptoms associated therewith be completely eliminated. Treatments according to the disclosure may be applied prophylactically (e.g., to a subject at risk of developing a disease or disorder), palliatively or remedially. Prophylactic treatments can be administered to a subject prior to onset of a sign or symptom, during early onset of a sign or symptom (e.g., upon initial signs and symptoms), or after an established development of a sign or symptom. Prophylactic administration can occur for several days to years prior to the manifestation of a symptom.

### 5.2. Methods

The disclosure provides new uses for compounds of Formula (I), tautomers thereof, and pharmaceutically acceptable salts of the foregoing. Compounds of Formula (I), tautomers thereof, and pharmaceutically acceptable salts of the foregoing are described in greater detail in Section 5.3.

In one aspect, the disclosure provides a method of activating AMPK and/or increasing the phosphorylated AMPK (pAMPK)/AMPK ratio in a cell by contacting a cell of a subject with an agent of the disclosure (e.g., sephin1) in an amount effective to activate AMPK and/or increase the pAMPK/AMPK ratio in the cell. The contacting of the cell with the agent can be in vivo, for example by orally administering an amount of the agent to the subject effective to activate AMPK and/or increase the pAMPK/AMPK ratio in one or more cell types, tissues, and/or organs of the subject, for example heart, liver, kidney, lung, skeletal muscle, or eye.

In one aspect, the disclosure provides a method of inhibiting CHOP and/or increasing the phosphorylated CHOP (pCHOP)/CHOP ratio in a cell by contacting a cell of a subject with an agent of the disclosure (e.g., sephin1) in an amount effective to inhibit CHOP and/or increase the pCHOP/CHOP ratio in the cell. The contacting of the cell with the agent can be in vivo, for example by orally administering an amount of the agent to the subject effective to inhibit CHOP and/or increase the pCHOP/CHOP ratio in one or more cell types, tissues, and/or organs of the subject, for example heart, liver, kidney, lung, skeletal muscle, or eye.

In another aspect, the disclosure provides a method of treating a subject having or at risk of disease or disorder treatable by AMPK activation and/or CHOP inhibition, the method comprising administering a therapeutically effective amount of an agent of the disclosure (e.g., sephin1) to the subject. In some embodiments, the amount of the agent administered is an amount effective to activate AMPK and/or inhibit CHOP in one or more cel tissues, and/or organs, for example heart, liver, kidney, lung, skeletal muscle, or eye.

In some embodiments, the amount of the agent administered is an amount effective to increase the pAMPK/AMPK ratio in one or more cell types, tissues, and/or organs, for example heart, liver, kidney, lung, skeletal muscle, or eye. In some embodiments, the amount of the agent administered is an amount effective to increase the pCHOP/CHOP ratio in one or more cell types, tissues, and/or organs, for example heart, liver, kidney, lung, skeletal muscle, or eye. AMPK, pAMPK, CHOP, and pCHOP levels and ratios can be evaluated, for example, by measuring their levels using standard laboratory techniques, e.g., by Western blotting, flow cytometry, or fluorescence microscopy.

Activation of AMPK can suppress fatty acid and cholesterol synthesis. Accordingly, in some embodiments, the amount of agent administered to a subject is an amount effective to suppress fatty acid synthesis. In some embodiments, the amount of the agent administered to a subject is an amount effective to suppress cholesterol synthesis. In some embodiments, the amount of the agent administered to a subject is an amount effective to lower a subject's cholesterol level, for example, lower serum and/or liver cholesterol levels.

Activation of AMPK can increase skeletal muscle glucose uptake. Accordingly, in some embodiments, the amount of agent administered to a subject is an amount effective to increase skeletal muscle glucose uptake in the subject. Skeletal muscle glucose uptake can be measured, for example, by measuring forearm glucose uptake.

Exemplary diseases and disorders treatable by AMPK activation and/or by inhibiting CHOP include metabolic syndrome, non-alcoholic fatty liver disease, nonalcoholic steatohepatitis (NASH), obesity, type 2 diabetes, insulin resistance, glucose intolerance, chronic pain, sarcopenia, a neuromuscular disorder, heart failure, cardiac hypertrophy, diabetic cardiomyopathy, cardiac reperfusion injury, chronic kidney disease, polycystic kidney disease, acute kidney injury, diabetic nephropathy, cardiovascular diseases, inflammatory bowel disease, arthritis, hypertension, peripheral vascular disease, nephrogenic diabetes insipidus, glaucoma, an eye disease or disorder, ocular neovascularization, β-hemoglobinopathy, cancer, and fibrosis. Further exemplary diseases and disorders treatable by AMPK activation and/or inhibiting CHOP include cancer cachexia, advanced cancer, mitochondrial diseases, and amyotrophic lateral sclerosis (ALS). Diseases and disorders treatable by AMPK activation and/or treatable by inhibiting CHOP are described, for example, in Steinberg & Carling, 2019, Nat. Rev. Drug Discov. 18(7):527-551; Li et al., 2015, Oncotarget 6(10):7365-7378; Yang et al., 2017, Frontiers in Immunology 8:1612; Dai et al., 2016, Circ Res. 119(10):1089-1100; US 2017/0020909; U.S. Pat. Nos. 10,016,486; 10,143,703; 10,596,144; WO 2007/097751; WO 2017/011917; WO 2011 and WO 2022/072397, the contents of which are incorporated herein by reference in their entireties.

Accordingly, in some embodiments, the subject administered an agent of the disclosure has or is at risk of developing a disease or disorder described in the preceding paragraph.

In some embodiments, the subject has or is at risk of metabolic syndrome.

In some embodiments, the subject has or is at risk of non-alcoholic fatty liver disease. nonalcoholic steatohepatitis (NASH).

In some embodiments, the subject has or is at risk of obesity.

In some embodiments, the subject has or is at risk of type 2 diabetes.

In some embodiments, the subject has or is at risk of insulin resistance.

In some embodiments, the subject has or is at risk of glucose intolerance.

In some embodiments, the subject has or is at risk of chronic pain, for example post-surgical pain.

In some embodiments, the subject has or is at risk of sarcopenia.

In some embodiments, the subject has or is at risk of a neuromuscular disorder, for example Duchenne muscular dystrophy or myotonic dystrophy type 1.

In some embodiments, the subject has or is at risk of heart failure.

In some embodiments, the subject has or is at risk of cardiac hypertrophy.

In some embodiments, the subject has or is at risk of diabetic cardiomyopathy.

In some embodiments, the subject has or is at risk of cardiac reperfusion injury.

In some embodiments, the subject has or is at risk of a kidney disease (e.g., chronic kidney disease or acute kidney injury).

In some embodiments, the subject has or is at risk of chronic kidney disease.

In some embodiments, the subject has or is at risk of polycystic kidney disease, for example autosomal dominant polycystic kidney disease.

In some embodiments, the subject has or is at risk of acute kidney injury (AKI).

In some embodiments, the subject has or is at risk of diabetic nephropathy.

In some embodiments, the subject has or is at risk of a cardiovascular d example atherosclerosis, coronary artery disease, peripheral artery disease, or cerebrovascular disease. In some embodiments, the amount of agent administered is effective to reduce the risk of and/or prevent atherosclerotic plaque rupture. Thus, in some embodiments, the method is a method of promoting atherosclerotic plaque stability. In some embodiments, the amount of agent administered is effective to inhibit initiation of or slow progression of atherosclerosis. Thus, in some embodiments, administration of an agent can reduce the likelihood of acute coronary heart disease and/or stroke.

In some embodiments, the subject has or is at risk of inflammatory bowel disease.

In some embodiments, the subject has or is at risk of arthritis.

In some embodiments, the subject has or is at risk of hypertension.

In some embodiments, the subject has or is at risk of peripheral vascular disease.

In some embodiments, the subject has or is at risk of nephrogenic diabetes insipidus.

In some embodiments, the subject has or is at risk of glaucoma.

In some embodiments, the subject has or is at risk of an eye disease or disorder, for example retinopathy, symptoms associated with microangiopathy, neovascular glaucoma, corneal graft rejection, glaucoma, herpetic and infectious keratitis, ocular ischemia, neovascular glaucoma, corneal, uveal and/or iris neovascularization, orbital and/or eyelid tumor(s), Stevens Johnson Syndrome, ocular cicatricial pemphigoid, injury, or ocular surface disease. In some embodiments, the eye disease or disorder is macular degeneration, for example wet age-related macular degeneration (AMD).

In some embodiments, the subject has or is at risk of ocular neovascularization, for example characterized by surface, corneal, retinal, choroidal, uveal, or iris neovascularization.

In some embodiments, the subject has or is at risk of β-hemoglobinopathy.

In some embodiments, the subject has or is at risk of cancer. In some embodiments, the cancer is hepatocellular carcinoma. In some embodiments, the cancer is lung cancer. In some embodiments, the cancer is colorectal cancer. In some embodiments, the cancer is liver cancer. In some embodiments, the cancer is melanoma. In some embodiments, the cancer is breast cancer. In some embodiments, the cancer is prostate cancer. In some embodiments, the cancer is ovarian cancer. In some embodiments, the cancer is leukemia.

In some embodiments, the subject has or is at risk of fibrosis. In some embodiments, the fibrosis is lung fibrosis. In some embodiments, the fibrosis is kidney fibrosis, for example glomerulosclerosis and/or tubulointerstitial fibrosis. In some embodiments, the fi fibrosis. In some embodiments, the fibrosis is cardiac fibrosis.

In some embodiments, the subject has cancer cachexia. In some embodiments, the subject has advanced cancer. In some embodiments, the subject has a mitochondrial disease.

In some embodiments, the subject has or is at risk of a disease or disorder associated with lipid hypermetabolism, for example cancer cachexia, advanced cancer, a mitochondrial disease, or ALS.

Agents of the disclosure can be administered to a subject by any suitable route. Suitable routes of administration include, but are not limited to, the oral route of administration. Other routes include topical (including dermal, buccal, ocular and sublingual), rectal or parenteral (including subcutaneous, intradermal, intramuscular and intravenous), nasal, intra-ocularly and pulmonary administration e.g., by inhalation. The most suitable route may depend upon the condition or disorder of the subject. In some embodiments, the agent is administered orally.

A person of ordinary skill in the art can determine an appropriate dose of an agent of the disclosure to administer to a subject. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. Exemplary dose ranges include the ranges 0.01 mg/kg to 100 mg/kg, 0.1 mg/kg to 20 mg/kg, 0.1 mg/kg to 50 mg/kg, and 0.1 mg/kg to 20 mg/kg. An exemplary daily dose range is 0.4 mg/kg/day to 400 mg/kg/day.

### 5.3. Agents and Compositions

Agents useful in the methods of the disclosure include compounds of Formula (I):

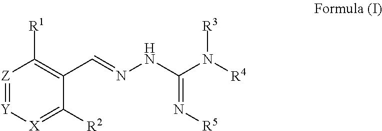

tautomers thereof, and pharmaceutically acceptable salts of the foregoing, wherein:
R¹ is alkyl, O-alkyl, Cl, F or Br;
R² is H or F;
R³ is selected H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl, and alkoxy;
X and Z are each independently CR¹¹;
Y is CR¹¹ or N; and
R¹¹ is H, alkyl or F.

Preferably, each alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

Preferably, each cycloalkyl group is a C₃₋₁₂ cycloalkyl group.

Preferably, each alkenyl group is a C₂₋₂₀ alkenyl group, a C₂₋₁₅ alkenyl group, a C₂₋₁₂ alkenyl group, a C₂₋₆ alkenyl group, or a C₂₋₃ alkenyl group.

Preferably, each “aryl” group is a C₆₋₁₂ aromatic group, for example phenyl or naphthyl.

In some embodiments, R¹ is Cl, Br, CH₃, H, or F.

In some embodiments, R² is H.

In some embodiments, Y is CR¹¹.

In some embodiments, X, Y, and Z are each CH.

In some embodiments, R³ and R⁴ are both H.

In some embodiments, R³ is H, R⁴ is C(O)R⁶, and R⁶ is CH₃ or OCH₃.

In some embodiments, the compound of Formula (I) has one of the following structures:

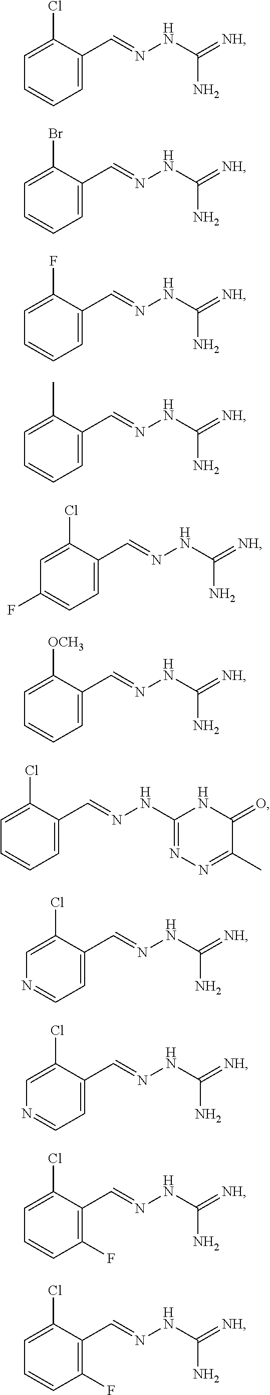

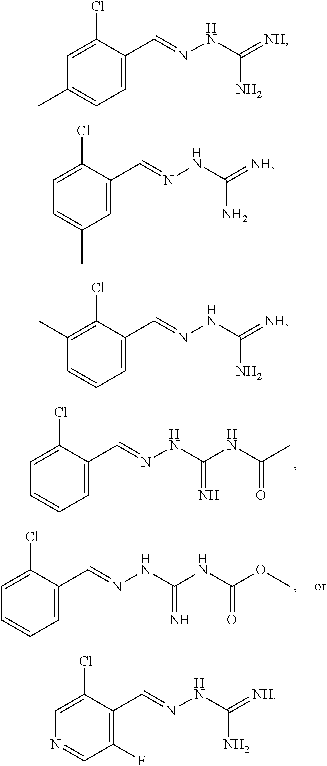

In some embodiments, the compound of Formula (I) has the structure:

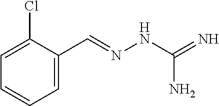 [SMILES: N=C(N)N/N=C/c1ccccc1Cl]

(corresponding to the structure of sephin1).

In some embodiments, the compound of Formula (I) has the structure:

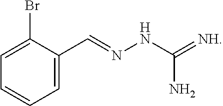 [SMILES: N=C(N)N/N=C/c1ccccc1Br]

In some embodiments, the compound of Formula (I) has the structure:

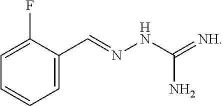 [SMILES: N=C(N)N/N=C/c1ccccc1F]

In some embodiments, the compound of Formula (I) has the structure:

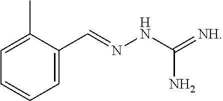

In some embodiments, the compound of Formula (I) has the structure:

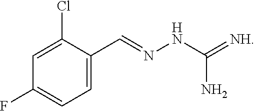 [SMILES: N=C(N)N/N=C/c1ccc(F)cc1Cl]

In some embodiments, the compound of Formula (I) has the structure:

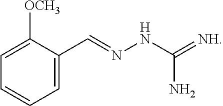 [SMILES: COc1ccccc1/C=N/NC(=N)N]

In some embodiments, the compound of Formula (I) has the structure:

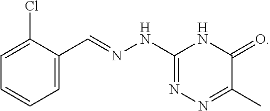 [SMILES: Cc1nnc(N/N=C/c2ccccc2Cl)[nH]c1=O]

In some embodiments, the compound of Formula (I) has the structure:

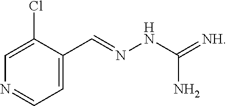 [SMILES: N=C(N)N/N=C/c1ccncc1Cl]

In some embodiments, the compound of Formula (I) has the structure:

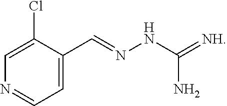 [SMILES: N=C(N)N/N=C/c1ccncc1Cl]

In some embodiments, the compound of Formula (I) has the structure:

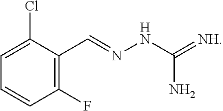 [SMILES: N=C(N)N/N=C/c1c(F)cccc1Cl]

In some embodiments, the compound of Formula (I) has the structure:

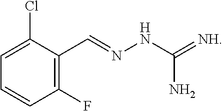 [SMILES: N=C(N)N/N=C/c1c(F)cccc1Cl]

In some embodiments, the compound of Formula (I) has the structure:

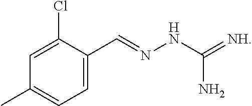

In some embodiments, the compound of Formula (I) has the structure:

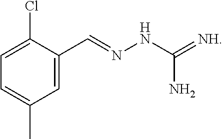

In some embodiments, the compound of Formula (I) has the structure:

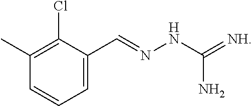

In some embodiments, the compound of Formula (I) has the structure:

In some embodiments, the compound of Formula (I) has the structure:

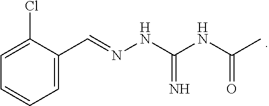 [SMILES: CC(=O)NC(=N)N/N=C/c1ccccc1Cl]

In some embodiments, the compound of Formula (I) has the structure:

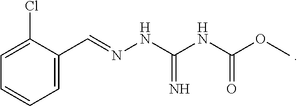 [SMILES: COC(=O)NC(=N)N/N=C/c1ccccc1Cl]

In some embodiments, the compound of Formula (I) has the structure:

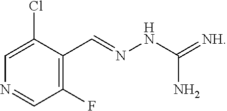 [SMILES: N=C(N)N/N=C/c1c(F)cncc1Cl]

Methods for synthesizing compounds of Formula (I) are known in the art. See, for example, WO 2016/001389, the contents of which are incorporated herein by reference in their entireties.

Those skilled in the art will appreciate that compounds of Formula (I) can undergo tautomerization. For example, the compound having the structure

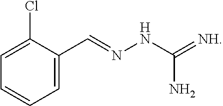 [SMILES: N=C(N)N/N=C/c1ccccc1Cl]

can convert to the tautomer

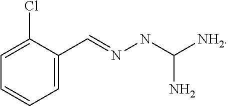 [SMILES: NC(N)N/N=C/c1ccccc1Cl]

Thus, tautomers of compounds of Formula (I) (including tautomeric mixtures) can be used in the methods and compositions of the disclosure.

Compounds of Formula (I) and tautomers thereof can be used in a salt form. Pharmaceutically acceptable salts that can be used include suitable acid addition or base salts thereof. A review of suitable pharmaceutical salts is found in Berge et al, 1977, J Pharm Sci 66:1-19.

Salts can be formed, for example with strong inorganic acids such as mineral acids, e.g. hydrohalic acids such as hydrochloric, hydrobromidic and hydroiodic acids, sulfuric acid, phosphoric acid sulphate, bisulphate, hemisulphate, thiocyanate, persulphate and sulphonic acids; with strongorganic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Exemplary salts include, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, tartrate, lactobionate, pivolate, camphorate, undecanoate, and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. In some embodiments, the salt is an acetic acid salt. In other embodiments, the salt is a carbonic acid salt.

In another aspect, the disclosure provides agents of the disclosure (e.g., sephin1) for use in treating a disease or disorder treatable by AMPK activation, for example a disease or disorder identified in Section 5.2.

In another aspect, the disclosure provides use of agents of the disclosure (e.g., sephin1) in the manufacture of a medicament for treating a disease or disorder treatable by AMPK activation, for example a disease or disorder identified in Section 5.2.

Agents of the disclosure (e.g., sephin1) can be formulated for the intended route of administration, for example according to techniques known in the art (e.g., as described in Allen et al., eds., 2012, Remington: The Science and Practice of Pharmacy, 22^{nd} Edition, Pharmaceutical Press, London, UK).

Agents of the disclosure (e.g., sephin1) can be formulated as a pharmaceutical composition comprising the agent and one or more pharmaceutical excipients, for example one or more excipients described in Handbook of Pharmaceutical Excipients, 8^{th} Revised Ed. (2017), incorporated by reference in its entirety. The pharmaceutical compositions can be presented in unit dosage form (e.g., containing from 1 mg to 250 mg, 10 mg to 100 mg, 1 mg to 100 mg, 2.5 mg to 60 mg, or 15 mg to 50 mg of an agent of the disclosure).

### 6. EXAMPLES

### 6.1. Example 1: AMPK Activating Activity of Sephin1

AMPK activating activity of sephin1 was evaluated in human kidney 2 (HK-2) cells. The HK-2 cell line is a proximal tubular cell line derived from normal kidney immortalized by transduction with human papilloma virus 16 (HPV-16) E6/E7 genes.

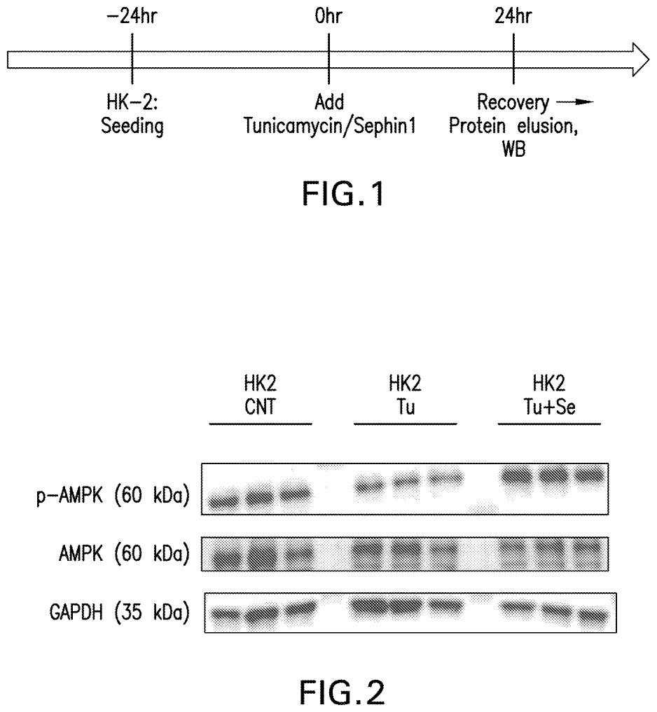

HK-2 cells were seeded in a six well plate at a cell density of 7×10⁴ cells per well 24 hours prior to the start of the study (T₋₂₄). At T₀, tunicamycin (a chemical ER stress-inducer) and sephin1 were added to the culture medium at a concentration of 5 μg/ml and 1 μM, respectively. 24 hours later (at T₂₄) cells were recovered and protein was analyzed by Western blot analysis. The timeline of the study is shown in FIG. 1.

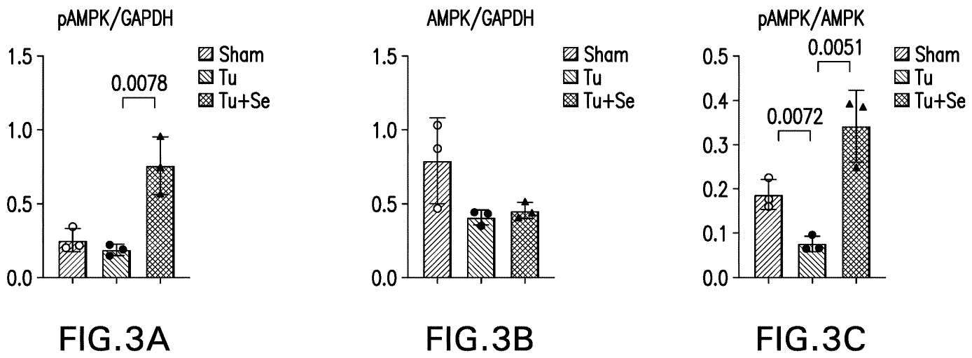

Western blots for phosphorylated AMPK (p-AMPK), AMPK, and GAPDH shown in FIG. 2. Ratios of pAMPK/GAPDH, AMPK/GAPDH, and pAMPK/AMPK are shown in FIGS. 3A-3C. As shown in FIG. 2 and FIG. 3A, pAMPK levels were strongly elevated in cells treated with sephin1 compared to untreated cells (sham) and cells treated with tunicamycin (Tu) alone. As shown in FIG. 2 and FIG. 3B, AMPK levels were not elevated by either tunicamycin or tunicamycin+sephin1 treatment; rather, AMPK levels were reduced compared to untreated cells. As shown in FIG. 2 and FIG. 3C, the pAMPK/AMPK ratio was greatly elevated in cells treated with sephin1 compared to untreated cells (sham) and cells treated with tunicamycin (Tu) alone. Thus, the data shows that sephin1 treatment resulted in activation of AMPK.

### 6.2. Example 2: Sephin1 Treatment Decreases CHOP Expression and Increases p-CHOP

The effect of sephin1 on CHOP expression and p-CHOP levels was evaluated.

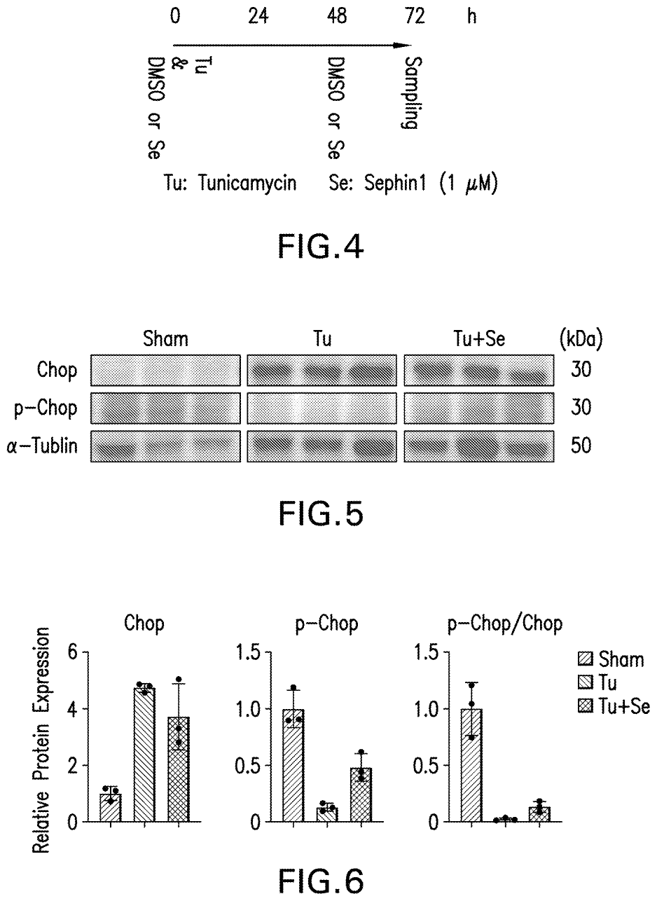

At the start of the study (T₀), cells were treated with tunicamycin (Tu) and either 1 μM sephin 1 (Se) or DMSO (sham). 24 hours and 48 hours later, cells were again treated with 1 μM sephin 1 (Se) or DMSO (sham). Sampling was performed at 72 hours. The study timeline is shown in FIG. 4.

Protein levels are shown in FIG. 5 and relative protein expression is shown in FIG. 6. Sephin1 treatment reduced CHOP levels and increased p-CHOP compared to tunicamycin only treatment (FIG. 5 and FIG. 6).

### 6.3. Example 3: Sephin1 Treatment of HK-2 Cells

The effect of sephin1 on CHOP expression and p-CHOP levels was evaluated in human kidney 2 (HK-2) cells.

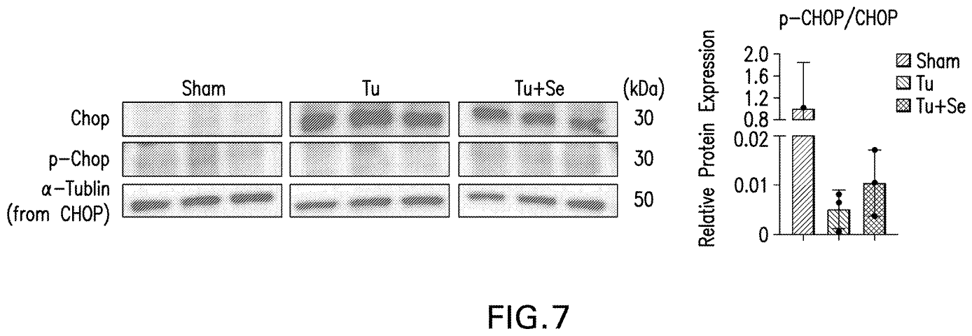

HK-2 cells were seeded one day prior to the start of the study (T_{-1day}). At T₀, tunicamycin and sephin1 were added to the culture medium. 24 hours later (at T₂₄) cells were recovered and protein levels were analyzed. The timeline of the study is shown in FIG. 7.

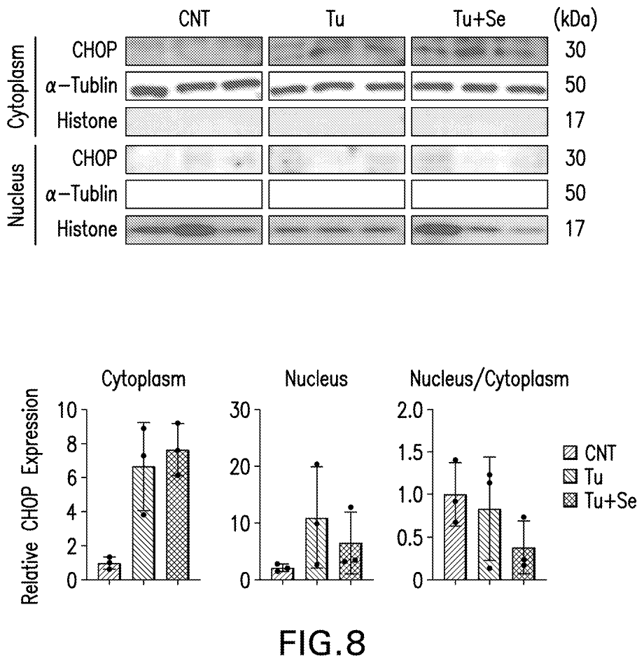

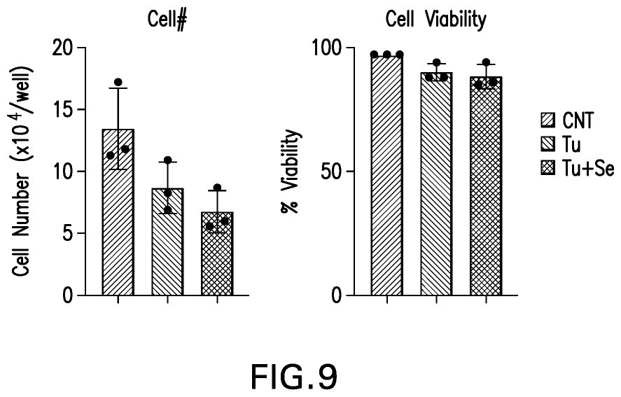

Protein levels are shown in FIG. 8 and FIG. 9. Cell number and cell viability are shown in FIG. 10. An increase in p-CHOP/CHOP ratio was observed in sephin1 treated cells compared to tunicamycin only treated cells (FIG. 8). An increase in CHOP expression in the cytoplasm, decreased CHOP expression in the nucleus, and a decreased nucleus/cytoplasm CHOP ratio was observed in sephin1 treated cells compared to tunicamycin only treated cells (FIG. 9).

### 7. SPECIFIC EMBODIMENTS

The present disclosure is exemplified by the specific embodiments below.

1. A method of activating AMP-activated protein kinase (AMPK) and/or increasing phosphorylated AMPK (pAMPK)/AMPK ratio, comprising contacting a cell of a subject with an agent which is a compound of Formula (I):

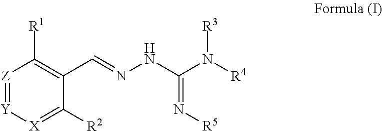

or a tautomer thereof, or a pharmaceutically acceptable salt of the foregoing, in an amount effective to activate AMPK and/or increase the pAMPK/AMPK ratio in the cell, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl, and alkoxy;
X and Z are each independently CR¹¹;
Y is CR¹¹ or N; and
R¹¹ is H, alkyl or F.

2. A method of inhibiting C/EBP homologous protein (CHOP) and/o phosphorylated a CHOP (pCHOP)/CHOP ratio in a cell, comprising contacting a cell of a subject with an agent which is a compound of Formula (I):

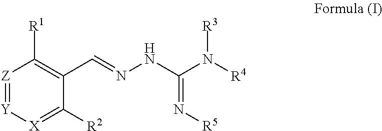

or a tautomer thereof, or a pharmaceutically acceptable salt of the foregoing, in an amount effective to inhibit C/EBP homologous protein (CHOP) and/or increase the pCHOP/CHOP ratio in the cell, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl, and alkoxy;
X and Z are each independently CR¹¹;
Y is CR¹¹ or N; and
R¹¹ is H, alkyl or F.

3. The method of embodiment 1 or embodiment 2, wherein the contacting is in vivo.

4. A method of treating a subject having or at risk of disease or disc treatable by (a) AMP-activated protein kinase (AMPK) activation and/or (b) inhibiting C/EBP homologous protein (CHOP), comprising administering to the subject a therapeutically effective amount of an agent which is a compound of Formula (I):

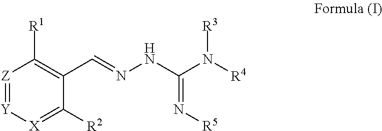

or a tautomer thereof, or a pharmaceutically acceptable salt of the foregoing, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy;
X and Z are each independently CR¹¹
Y is CR¹¹ or N; and
R¹¹ is H, alkyl or F.

5. The method of any one of embodiments 2 to 4, wherein the amount of the agent is an amount effective to activate AMPK in one or more cell types, tissues, and/or organs.

6. The method of any one of embodiments embodiment 2 to 5, whe amount of the agent is an amount effective to increase the pAMPK/AMPK ratio in one or more cell types, tissues, and/or organs.

7. The method of any one of embodiments 2 to 6, wherein the amount of the agent in an amount effective to suppress fatty acid and/or cholesterol synthesis in the subject.

8. The method of any one of embodiments 2 to 7, wherein the amount of the agent in an amount effective to lower serum and/or liver cholesterol levels.

9. The method of any one of embodiments 2 to 8, wherein the amount of the agent in an amount effective to increase skeletal muscle glucose uptake.

10. The method of any one of embodiments 2 to 9, wherein the amount of the agent in an amount effective to inhibit CHOP in one or more cell types, tissues, and/or organs.

11. The method of any one of embodiments 2 to 10, wherein the amount of the agent in an amount effective to increase the pCHOP/CHOP ratio in one or more cell types, tissues, and/or organs.

12. The method of any one of embodiments 1 to 11, wherein the subject has or is at risk of a disease or disorder which is metabolic syndrome, non-alcoholic fatty liver disease, nonalcoholic steatohepatitis (NASH), obesity, type 2 diabetes, insulin resistance, glucose intolerance, chronic pain, sarcopenia, a neuromuscular disorder, heart failure, cardiac hypertrophy, diabetic cardiomyopathy, cardiac reperfusion injury, chronic kidney disease, polycystic kidney disease, diabetic nephropathy, a cardiovascular disease, inflammatory bowel disease, arthritis, hypertension, peripheral vascular disease, nephrogenic diabetes insipidus, glaucoma, an eye disease or disorder, ocular neovascularization, β-hemoglobinopathy, cancer, fibrosis, cancer cachexia, or a mitochondrial disease.

13. The method of any one of embodiments 1 to 11, wherein the subject has or is at risk of a disease or disorder which is metabolic syndrome, non-alcoholic fatty liver disease, nonalcoholic steatohepatitis (NASH), obesity, type 2 diabetes, insulin resistance, glucose intolerance, chronic pain, sarcopenia, a neuromuscular disorder, heart failure, cardiac hypertrophy, diabetic cardiomyopathy, cardiac reperfusion injury, chronic kidney disease, polycystic kidney disease, acute kidney injury, diabetic nephropathy, a cardiovascular disease, inflammatory bowel disease, arthritis, hypertension, per vascular disease, nephrogenic diabetes insipidus, glaucoma, an eye disease or disorder, ocular neovascularization, β-hemoglobinopathy, cancer, fibrosis, cancer cachexia, or a mitochondrial disease.

14. The method of any one of embodiments 1 to 11, wherein the subject has or is at risk of a disease or disorder associated with lipid hypermetabolism.

15. The method of embodiment 12 or embodiment 13, wherein the disease or disorder is metabolic syndrome.

16. The method of embodiment 12 or embodiment 13, wherein the disease or disorder is non-alcoholic fatty liver disease.

17. The method of embodiment 12 or embodiment 13, wherein the disease or disorder is nonalcoholic steatohepatitis (NASH).

18. The method of embodiment 12 or embodiment 13, wherein the disease or disorder is obesity.

19. The method of embodiment 12 or embodiment 13, wherein the disease or disorder is type 2 diabetes.

20. The method of embodiment 12 or embodiment 13, wherein the disease or disorder is insulin resistance.

21. The method of embodiment 12 or embodiment 13, wherein the disease or disorder is glucose intolerance.

22. The method of embodiment 12 or embodiment 13, wherein the disease or disorder is chronic pain.

23. The method of embodiment 22, wherein the chronic pain is post-surgical pain.

24. The method of embodiment 12 or embodiment 13, wherein the disease or disorder is sarcopenia.

25. The method of embodiment 12 or embodiment 13, wherein the disease or disorder is a neuromuscular disorder.

26. The method of embodiment 25, wherein the neuromuscular disoi Duchenne muscular dystrophy or myotonic dystrophy type 1.

27. The method of embodiment 12 or embodiment 13, wherein the disease or disorder is heart failure.

28. The method of embodiment 12 or embodiment 13, wherein the disease or disorder is cardiac hypertrophy.

29. The method of embodiment 12 or embodiment 13, wherein the disease or disorder is diabetic cardiomyopathy.

30. The method of embodiment 12 or embodiment 13, wherein the disease or disorder is cardiac reperfusion injury.

31. The method of embodiment 12 or embodiment 13, wherein the disease or disorder is chronic kidney disease.

32. The method of embodiment 12 or embodiment 13, wherein the disease or disorder is polycystic kidney disease

33. The method of embodiment 32, wherein the polycystic kidney disease is autosomal dominant polycystic kidney disease.

34. The method of embodiment 12 or embodiment 13, wherein the disease or disorder is diabetic nephropathy.

35. The method of embodiment 12 or embodiment 13, wherein the disease or disorder is a cardiovascular disease.

36. The method of embodiment 35, wherein the cardiovascular disease is atherosclerosis.

37. The method of embodiment 35, wherein the cardiovascular disease is coronary artery disease.

38. The method of embodiment 35, wherein the cardiovascular disease is peripheral artery disease.

39. The method of embodiment 35, wherein the cardiovascular disease is cerebrovascular disease.

40. The method of embodiment 12 or embodiment 13, wherein the d disorder is inflammatory bowel disease.

41. The method of embodiment 12 or embodiment 13, wherein the disease or disorder is arthritis.

42. The method of embodiment 12 or embodiment 13, wherein the disease or disorder is hypertension.

43. The method of embodiment 12 or embodiment 13, wherein the disease or disorder is peripheral vascular disease.

44. The method of embodiment 12 or embodiment 13, wherein the disease or disorder is nephrogenic diabetes insipidus.

45. The method of embodiment 12 or embodiment 13, wherein the disease or disorder is an eye disease or disorder.

46. The method of embodiment 45, wherein the eye disease or disorder is retinopathy, symptoms associated with microangiopathy, neovascular glaucoma, corneal graft rejection, glaucoma, herpetic and infectious keratitis, ocular ischemia, neovascular glaucoma, corneal, uveal and/or iris neovascularization, orbital and/or eyelid tumor(s), Stevens Johnson Syndrome, ocular cicatricial pemphigoid, injury, or ocular surface disease.

47. The method of embodiment 45, wherein the disease or disorder is macular degeneration.

48. The method of embodiment 46, wherein the disease or disorder is wet age-related macular degeneration (AMD).

49. The method of embodiment 12 or embodiment 13, wherein the disease or disorder is ocular neovascularization.

50. The method of embodiment 45, wherein the neovascularization is characterized by surface, corneal, retinal, choroidal, uveal, or iris neovascularization.

51. The method of embodiment 12 or embodiment 13, wherein the disease or disorder is glaucoma.

52. The method of any one of embodiments 2 to 51, wherein the amount of the agent is an amount effective to reduce intraocular pressure.

53. The method of embodiment 12 or embodiment 13, wherein the d disorder is β-hemoglobinopathy.

54. The method of embodiment 12 or embodiment 13, wherein the disease or disorder is cancer, optionally wherein the cancer is advanced cancer.

55. The method of embodiment 54, wherein the cancer is hepatocellular carcinoma.

56. The method of embodiment 54, wherein the cancer is lung cancer.

57. The method of embodiment 54, wherein the cancer is colorectal cancer.

58. The method of embodiment 54, wherein the cancer is liver cancer.

59. The method of embodiment 54, wherein the cancer is melanoma.

60. The method of embodiment 54, wherein the cancer is breast cancer.

61. The method of embodiment 54, wherein the cancer is prostate cancer.

62. The method of embodiment 54, wherein the cancer is ovarian cancer.

63. The method of embodiment 54, wherein the cancer is leukemia.

64. The method of embodiment 12 or embodiment 13, wherein the disease or disorder is fibrosis.

65. The method of embodiment 64, wherein the fibrosis is lung fibrosis.

66. The method of embodiment 64, wherein the fibrosis is kidney fibrosis.

67. The method of embodiment 66, wherein the fibrosis is glomerulosclerosis and/or tubulointerstitial fibrosis.

68. The method of embodiment 66 or embodiment 67, wherein the kidney fibrosis is kidney fibrosis resulting from chronic kidney disease (CKD), optionally wherein the CKD is hypertensive CKD.

69. The method of embodiment 64, wherein the fibrosis is liver fibrosis.

70. The method of embodiment 64, wherein the fibrosis is cardiac fibrosis.

71. The method of embodiment 12 or embodiment 13, wherein the d disorder is cancer cachexia.

72. The method of embodiment 12 or embodiment 13, wherein the disease or disorder is a mitochondrial disease.

73. The method of embodiment 13, of any one of embodiments 1 to 11, wherein the subject has or is at risk of a disease or disorder which is a kidney disease or disorder.

74. The method of embodiment 13 or 73, wherein the disease or disorder is acute kidney injury.

75. The method of any one of embodiments 12 to 74, wherein the subject has the disease or disorder.

76. The method of any one of embodiments 12 to 74, wherein the subject is at risk of the disease or disorder.

77. The method of any one of embodiments 1 to 76, wherein R¹ is Cl, Br, CH₃, H, or F.

78. The method of embodiment 77, wherein R¹ is Cl.

79. The method of any one of embodiments 1 to 78, wherein R² is H.

80. The method of any one of embodiments 1 to 79, wherein Y is CR¹¹.

81. The method of any one of embodiments 1 to 80, wherein X, Y, and Z are each CH.

82. The method of any one of embodiments 1 to 81, wherein R³ and R⁴ are both H.

83. The method of any one of embodiments 1 to 81, wherein R³ is H, R⁴ is C(O)R⁶, and R⁶ is CH₃ or OCH₃.

84. The method of any one of embodiments 1 to 76, wherein the agent is

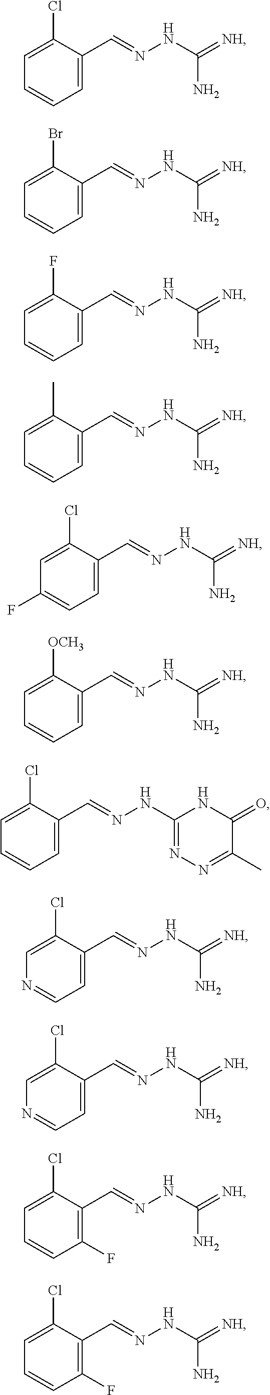

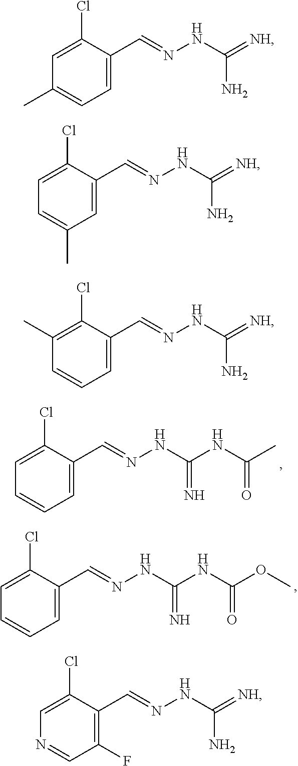

or a tautomer thereof, or a pharmaceutically acceptable salt of the foregoing.

85. The method of embodiment 84, wherein the agent is

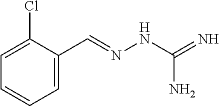 [SMILES: N=C(N)N/N=C/c1ccccc1Cl]

or a tautomer thereof, or a pharmaceutically acceptable salt of the foregoing.

86. The method of embodiment 84, wherein the agent is

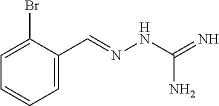 [SMILES: N=C(N)N/N=C/c1ccccc1Br]

or a tautomer thereof, or a pharmaceutically acceptable salt of the foregoing.

87. The method of embodiment 84, wherein the agent is

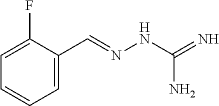 [SMILES: N=C(N)N/N=C/c1ccccc1F]

or a tautomer thereof, or a pharmaceutically acceptable salt of the foregoing.

88. The method of embodiment 84, wherein the agent is

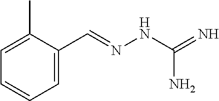

or a tautomer thereof, or a pharmaceutically acceptable salt of the foregoing.

89. The method of embodiment 84, wherein the agent is

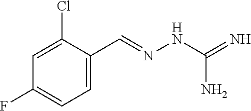 [SMILES: N=C(N)N/N=C/c1ccc(F)cc1Cl]

or a tautomer thereof, or a pharmaceutically acceptable salt of the foregoing.

90. The method of embodiment 84, wherein the agent is

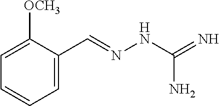 [SMILES: COc1ccccc1/C=N/NC(=N)N]

or a tautomer thereof, or a pharmaceutically acceptable salt of the foregoing.

91. The method of embodiment 84, wherein the agent is

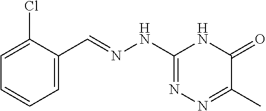 [SMILES: Cc1nnc(N/N=C/c2ccccc2Cl)[nH]c1=O]

or a tautomer thereof, or a pharmaceutically acceptable salt of the foregoing.

92. The method of embodiment 84, wherein the agent is

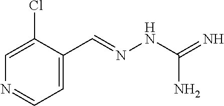 [SMILES: N=C(N)N/N=C/c1ccncc1Cl]

or a tautomer thereof, or a pharmaceutically acceptable salt of the foregoing.

93. The method of embodiment 84, wherein the agent is

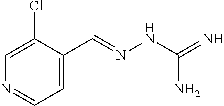 [SMILES: N=C(N)N/N=C/c1ccncc1Cl]

or a tautomer thereof, or a pharmaceutically acceptable salt of the foregoing.

94. The method of embodiment 84, wherein the agent is

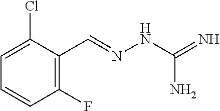 [SMILES: N=C(N)N/N=C/c1c(F)cccc1Cl]

or a tautomer thereof, or a pharmaceutically acceptable salt of the foregoing.

95. The method of embodiment 84, wherein the agent is

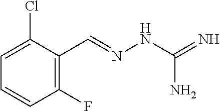 [SMILES: N=C(N)N/N=C/c1c(F)cccc1Cl]

or a tautomer thereof, or a pharmaceutically acceptable salt of the foregoing.

96. The method of embodiment 84, wherein the agent is

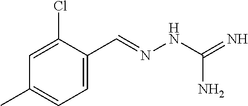

or a tautomer thereof, or a pharmaceutically acceptable salt of the foregoing.

97. The method of embodiment 84, wherein the agent is

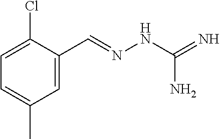

or a tautomer thereof, or a pharmaceutically acceptable salt of the foregoing.

98. The method of embodiment 84, wherein the agent is

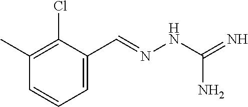

or a tautomer thereof, or a pharmaceutically acceptable salt of the foregoing.

99. The method of embodiment 84, wherein the agent is

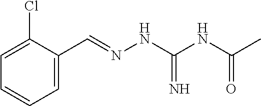 [SMILES: CC(=O)NC(=N)N/N=C/c1ccccc1Cl]

or a tautomer thereof, or a pharmaceutically acceptable salt of the foregoing.

100. The method of embodiment 84, wherein the agent is

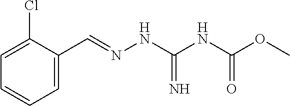 [SMILES: COC(=O)NC(=N)N/N=C/c1ccccc1Cl]

or a tautomer thereof, or a pharmaceutically acceptable salt of the foregoing.

101. The method of embodiment 84, wherein the agent is

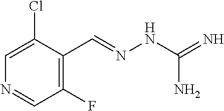 [SMILES: N=C(N)N/N=C/c1c(F)cncc1Cl]

or a tautomer thereof, or a pharmaceutically acceptable salt of the foregoing.

102. The method of any one of embodiments 1 to 101, wherein the agent is a pharmaceutically acceptable salt of a compound of Formula (I) or tautomer thereof.

103. The method of embodiment 102, wherein the salt is an acetic acid salt.

104. The method of embodiment 102, wherein the salt is a carbonic acid salt.

105. The method of any one of embodiments 1 to 104, wherein the agent is administered to the subject orally.

106. An agent as described in any one of the preceding embodiments for use in a method of any one of the preceding embodiments.

107. A pharmaceutical composition comprising an agent as described in any one of the preceding embodiments and a pharmaceutically acceptable excipient for use in a method of any one of the preceding embodiments.

108. Use of an agent as described in any one of the preceding embodiments for the manufacture of a medicament for treating a disease or disorder described in any of the preceding embodiments.

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A method of activating AMP-activated protein kinase (AMPK) and/or increasing phosphorylated AMPK (pAMPK)/AMPK ratio, comprising contacting a cell of a subject with an agent which is a compound of Formula (I): 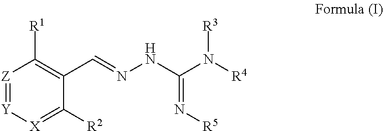
or a tautomer thereof, or a pharmaceutically acceptable salt of the foregoing, in an amount effective to activate AMPK and/or increase the pAMPK/AMPK ratio in the cell, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl, and alkoxy;
X and Z are each independently CR¹¹;
Y is CR¹¹ or N; and
R¹¹ is H, alkyl or F.

2. A method of inhibiting C/EBP homologous protein (CHOP) and/or increasing phosphorylated a CHOP (pCHOP)/CHOP ratio in a cell, comprising contacting a cell of a subject with an agent which is a compound of Formula (I): 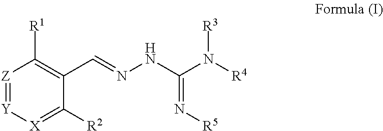
or a tautomer thereof, or a pharmaceutically acceptable salt of the foregoing, in an amount effective to inhibit C/EBP homologous protein (CHOP) and/or increase the pCHOP/CHOP ratio in the cell, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl, and alkoxy;
X and Z are each independently CR¹¹;
Y is CR¹¹ or N; and
R¹¹ is H, alkyl or F.

3. The method of claim 1 or claim 2, wherein the contacting is in vivo.

4. A method of treating a subject having or at risk of disease or disorder treatable by (a) AMP-activated protein kinase (AMPK) activation and/or (b) inhibiting C/EBP homologous protein (CHOP), comprising administering to the subject a therapeutically effective amount of an agent which is a compound of Formula (I): 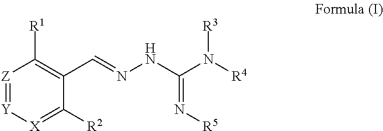
or a tautomer thereof, or a pharmaceutically acceptable salt of the foregoing, wherein:
R¹ is alkyl, O-alkyl, Cl, F or Br;
R² is H or F;
R³ is H or alkyl;
R⁴ is H or C(O)R⁶;
R⁵ is H;
or R⁴ and R⁵ are linked to form a 5 to 6 membered saturated or unsaturated heterocyclic group optionally comprising 1 or 2 heteroatoms in addition to the N atoms to which R⁴ and R⁵ are bound, and where said heterocyclic group is optionally substituted with one or more R¹⁰ groups;
R⁶ is selected R⁷, OR⁷ or NR⁸R⁹;
R⁷, R⁸ and R⁹ are each independently selected from alkyl, cycloalkyl, aralkyl, cycloalkenyl, heterocyclyl and aryl, each of which is optionally substituted with one or more R¹⁰ groups;
each R¹⁰ is independently selected from halogen, OH, ═O, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy;
X and Z are each independently CR¹¹
Y is CR¹¹ or N; and
R¹¹ is H, alkyl or F.

5. The method of any one of claims 2 to 4, wherein the amount of the agent is an amount effective to:
(a) activate AMPK in one or more cell types, tissues, and/or organs; and/or
(b) increase the pAMPK/AMPK ratio in one or more cell types, tissues, and/or organs; and/or
(c) suppress fatty acid and/or cholesterol synthesis in the subject; and/or
(d) lower serum and/or liver cholesterol levels; and/or
(e) increase skeletal muscle glucose uptake; and/or
(f) inhibit CHOP in one or more cell types, tissues, and/or organs; and/or
(g) increase the pCHOP/CHOP ratio in one or more cell types, tissues, and/or organs.

6. The method of any one of claims 1 to 5, wherein the subject has or is at risk of a disease or disorder which is metabolic syndrome, non-alcoholic fatty liver disease, nonalcoholic steatohepatitis (NASH), obesity, type 2 diabetes, insulin resistance, glucose intolerance, chronic pain, sarcopenia, a neuromuscular disorder, heart failure, cardiac hypertrophy, diabetic cardiomyopathy, cardiac reperfusion injury, chronic kidney disease, polycystic kidney disease, diabetic nephropathy, a cardiovascular disease, inflammatory bowel disease, arthritis, hypertension, peripheral vascular disease, nephrogenic diabetes insipidus, glaucoma, an eye disease or disorder, ocular neovascularization, β-hemoglobinopathy, cancer, fibrosis, cancer cachexia, or a mitochondrial disease.

7. The method of any one of claims 1 to 5, wherein the subject has or is at risk of a disease or disorder associated with lipid hypermetabolism.

8. The method of any one of claims 1 to 5, wherein the subject has or is at risk of a disease or disorder which is a kidney disease or disorder.

9. The method of any one of claims 1 to 5, wherein the subject has or is at risk of acute kidney injury.

10. The method of any one of claims 6 to 9, wherein the subject has the disease or disorder.

11. The method of any one of claims 6 to 9, wherein the subject is at risk of the disease or disorder.

12. The method of any one of claims 1 to 11, wherein R¹ is Cl, Br, CH₃, H, or F.

13. The method of claim 12, wherein R¹ is Cl.

14. The method of any one of claims 1 to 13, wherein R² is H.

15. The method of any one of claims 1 to 14, wherein Y is CR¹¹.

16. The method of any one of claims 1 to 15, wherein X, Y, and Z are each CH.

17. The method of any one of claims 1 to 16, wherein R³ and R⁴ are

18. The method of any one of claims 1 to 16, wherein R³ is H, R⁴ is C(O)R⁶, and R⁶ is CH₃ or OCH₃.

19. The method of any one of claims 1 to 11, wherein the agent is 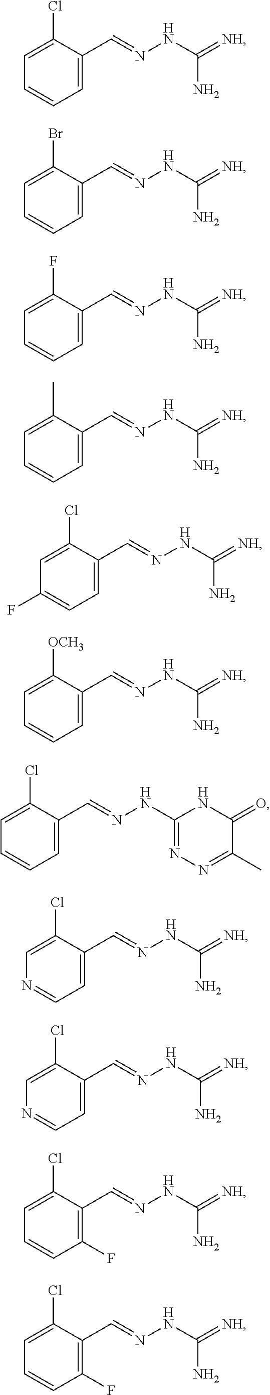 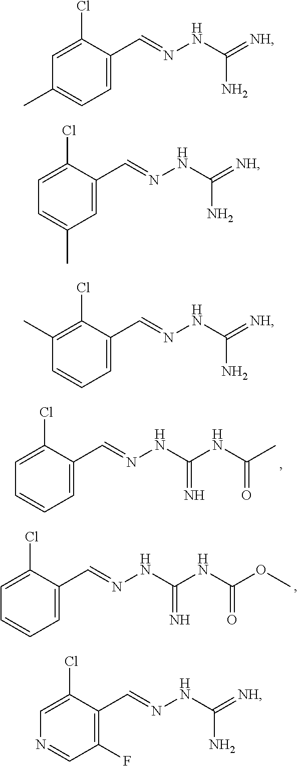
or a tautomer thereof, or a pharmaceutically acceptable salt of the foregoing.

20. The method of claim 19, wherein the agent is 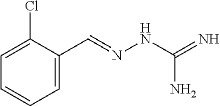 [SMILES: N=C(N)N/N=C/c1ccccc1Cl]
or a tautomer thereof, or a pharmaceutically acceptable salt of the foregoing.

21. The method of any one of claims 1 to 20, wherein the agent is a pharmaceutically acceptable salt of a compound of Formula (I) or tautomer thereof.

22. The method of claim 21, wherein the salt is an acetic acid salt.

23. The method of claim 21, wherein the salt is a carbonic acid salt.

24. The method of any one of claims 1 to 23, wherein the agent is administered to the subject orally.
